(19) United States
(12) Patent Application Publication
(10) Pub. No.: US 2002/0081594 A1
(43) Pub. Date: Jun. 27, 2002

(54) Uracil DNA metabolism as a target for chemotherapy: screening assays and related methods

(76) Inventors: Robert D. Ladner, Haddenfield, NJ (US); Salvatore J. Caradonna, Voorhees, NJ (US)

Correspondence Address: Saul Ewing LLP, Centre Square West, 38th Floor, 1500 Market Street, Philadelphia, PA, 19102-2186, US

(21) Appl. No.: 09/804,700

(22) Filed: Mar. 13, 2001

Related U.S. Application Data
(60) Provisional application No. 60/189,516, filed on Mar. 15, 2000.

(51) Int. Cl. C12Q 1/68

(52) U.S. Cl. 435/6

(57) ABSTRACT

A screening and development of agents that target and disrupt DNA replication is provided. In particular, aberrant UT1 metabolism is provided as a mechanism of inducing cytotoxicity and/or cytostasis.

### SCREENING ASSAYS AND RELATED METHODS

This application claims the benefit of priority of U.S. Provisional Application No. 60/189,516, filed on Mar. 15, 2000, the contents of which are incorporated herein by reference in its entirety.

### FIELD OF THE INVENTION

The invention relates to biotechnology, and to DNA and chemotherapeutic agents. More specifically, the invention relates to the screening and development of agents that target and disrupt DNA replication. In particular, the invention relates to aberrant uracil-DNA metabolism as a mechanism of inducing cytotoxicity and/or cytostasis.

### BACKGROUND OF THE INVENTION

Various scientific and scholarly articles are referenced in parentheses throughout the specification. These articles are incorporated by reference herein to describe the state of the art to which this invention pertains.

Thymidylate metabolism has long been an important target for widely utilized chemotherapeutic agents (e.g. the antifolates and fluoropyrimidines) that provide benefit in the treatment of leukemias, head and neck, breast and gastrointestinal cancers (Moertel, C. G. (1994) N. Engl. J. Med. 330: 1136-1143). The major mechanism of action of this class of antineoplastic drugs is the inhibition of enzymes that mediate critical steps in thymidylate metabolism. The de novo biosynthesis of TMP occurs by the reductive methylation of dUMP by the enzyme thymidylate synthase (TS) to yield TMP, which is then converted to TTP for DNA replication (FIG. 1A).

The methyl donor in TMP biosynthesis, 5, 10 methelenetetrahydrofolate (MTHF), is oxidized to dihydrofolate (DHF) so that the TS reaction constitutes a significant drain on cellular tetrahydrofolate (THF) pools. The levels of MTHF are maintained during TMP synthesis by the combined actions of dihydrofolate reductase (DHFR) and serine hydroxymethyltransferase (FIG. 1A). Chemotherapeutic agents such as 5-fluorouracil (5FU) and fluorodeoxyuridine (FUDR) block TMP biosynthesis by inhibiting TS directly. Inhibitors of DHFR (e.g. aminopterin/methotrexate) indirectly block TMP production by limiting the availability of MTHF (see FIG. 1B).

Studies attempting to elucidate the molecular mechanisms of cell killing mediated by inhibitors of TS and DHFR suggest that cytotoxicity results from a process termed “thymineless death”. Classically, cell death initiated by thymineless conditions has been described as simply the result of DNA synthesis arrest and DNA degradation due to extreme TTP pool depletion (Cohen, S. S. (1971) Ann. NY Acad. Sci. 106: 292-301). However, more recent investigations suggest that multiple factors contribute to the underlying mechanism of thymineless death, particularly imbalance of other dNTP pools. There is now limited evidence suggesting that elevated dUTP pools and misincorporation of uracil into DNA may play a significant role in initiating DNA damage and cell death in response to inhibition of de novo thymidylate metabolism (Aherne, G. W. and Browne S. (1999) Anticancer Drug Development Guide: Antifolate Drugs in Cancer Therapy. 409-421 Humana Press Inc., Towata, N.J.). Due to these findings, there is growing interest in the role of dUTP metabolism as a mediator of cytotoxicity and as a determinant of efficacy in the clinical use of anti-thymidylate chemotherapeutics. However, precise mechanistic studies have not been performed to assess the role of uracil-DNA metabolism in mediating cell death.

In virtually all known organisms, uracil is not a native component of DNA. However, uracil can arise in DNA either by the spontaneous deamination of cytosine residues or through dUTP utilization by DNA polymerases during replication (Lindahl, T. (1993) Nature 362: 709-715). Because cytosine deamination can lead to G:C to A:T transition mutations, the cell has evolved highly efficient mechanisms to facilitate the exclusion of uracil from DNA (Lindahl, T. (1982) Annu. Rev. Biochem. 51: 61-87). When uracil does occur in DNA, uracil-DNA glycosylase (UDG) initiates the base-excision repair pathway to remove and correct the misincorporated nucleotide. In order to prevent dUTP utilization during DNA replication, the enzyme deoxyuridine pyrophosphatase (dUTPase) hydrolyzes dUTP to yield dUMP and pyrophosphate. This reaction effectively eliminates dUTP from the DNA biosynthetic pathway and also provides substrate (dUMP) for the de novo synthesis of thymidylate (FIG. 1A). Therefore, under normal cellular conditions, the maintenance of uracil-free DNA is achieved through the combined actions of dUTPase and UDG.

Although dUTP is a normal intermediate in thymidylate biosynthesis, its extensive accumulation and misincorporation into DNA is lethal in both prokaryotic and eukaryotic organisms (El-Hajj, H. H., et al. (1988) J. Bacteriol. 170: 1069-1075; Gadsden, M. H., et al., (1993) EMBO J. 12: 4425-4431). The exact biochemical basis for uracil-DNA mediated cell death has not been definitively proven, however there is evidence suggesting that UDG-initiated repair may be a component of this process. For example, inactivation of dUTPase in Escherichia coli results in the dramatic accumulation of dUTP pools leading to extensive uracil misincorporation during replication. Under conditions of elevated dUTP pools, the cell engages in repeated cycles of uracil misincorporation and UDG-mediated repair. This iterative process results in increased recombination, DNA strand breaks, and ultimately cell death (El-Hajj, et al, supra). A similar phenomenon is thought to occur during inhibition of de novo thymidylate metabolism by anti cancer agents. Inhibition of the TS reaction leads to the accumulation of cellular dUMP pools and, as a result of mono- and di-phosphate kinases, induces a dramatic increase in dUTP pools. Once levels of dUTP accumulate beyond a threshold level, overwhelming cellular dUTPase activity, the dUTP/TTP ratio increases. Under these conditions, dUTP is misincorporated into replicating DNA resulting in uracil-DNA-mediated cytotoxicity (FIG. 1B). Implicit to this model of cell killing, are the central roles of dUTPase and uracil-DNA glycosylase.

The ubiquitous enzyme dUTPase is essential for viability in both prokaryotic and eukaryotic organisms. As the main regulator of dUTP pools, the expression of dUTPase could have profound effects on the utility of chemotherapeutics that inhibit thymidylate biosynthesis. Normally, dUTPase mediates a protective role by limiting the expansion of dUTP pools and countering the cytotoxic effect of uracil misincorporation. According to this model, elevated levels of dUTPase would prevent the accumulation of dUTP required for cell killing. To test this hypothesis, Canman and co-workers ectopically over expressed the E. coli dUTPase in a FUdR- sensitive human colorectal tumor cell line (HT29) and measured the response to the TS inhibitor FUdR. The manipulated cell lines (containing dUTPase activity 4 to 5-fold higher than controls) were protected from FUdR-induced DNA strand breaks and showed an increased viability over control cells (Canman, C. E., et al. (1994) Cancer Res. 54: 2296-2298). This study provided the first evidence in implicating dUTPase enzyme levels as an important factor in determining the efficacy of TS inhibition. Although the contribution of uracil-DNA-mediated cytotoxicity toward overall cell death is likely to vary between different cell lines, these data support the role of dUTP pool imbalance and uracil misincorporation as a contributing mechanism of TS inhibitor-based cytotoxicity.

Uracil-DNA glycosylase (UDG) is the first enzyme in the uracil-base excision repair pathway. By virtue of its central role in uracil-DNA repair, it is thought to be critical in initiating DNA strand breaks resulting from uracil misincorporation. Investigation of UDG in E. coli suggests that there is a direct relationship between UDG activity and uracil-DNA-mediated cytotoxicity. One study found that mutants of the UDG gene (ung-) could suppress lethality induced by mutations of the dUTPase gene (dut-) (Taylor and Weiss, (1982) J. Bacteriol. 151: 351-357). The dut-/ung- double mutants were able to replace thymidine in their DNA with up to ˜20% uracil (Warner, et al., (1981) J. Bacteriol. 145: 687-695). These and other investigations suggest that there is an upper limit of tolerable uracil substitution in DNA (20%) beyond which cells are inviable by an as-yet unidentified mechanism unrelated to DNA repair. Investigation of uracil-DNA metabolism in S. cerevisiae also support this theory, where dut-/ung- double mutants starved for thymidine appear to arrest in all phases of the cell cycle suggesting a general failure in transcription, however this has yet to be definitively proven. Consistent with this hypothesis, the transcription factor Gcn4 does not efficiently bind to its target sequence when uracil is substituted for thymidine (Pu and Struhl (1992) Nucleic Acids Res. 20: 771-775).

### SUMMARY OF THE INVENTION

In accordance with the present invention, it has now been discovered that aberrant uracil-DNA metabolism is the primary mediator of cytotoxicity induced by chemotherapeutic agents that inhibit de novo thymidylate metabolism in the yeast Saccharomyces cerevisiae. Utilizing a yeast model system, the inventors have demonstrated that aberrant uracil-DNA metabolism activates three distinct cell cycle checkpoints under different conditions. Based on this new evidence, the present invention provides a rapid screening method for chemotherapeutic agents that induce uracil misincorporation into DNA. Additional related methods are also provided.

The inventive method is directed to determining if a test compound induces uracil misincorporation into DNA. This method comprises providing aliquots of cells, exposing the cells to an agent that directly or indirectly inhibits thymidylate metabolism, in the presence or absence of the test compound, measuring one or more features of the exposed cells, and interpreting the measured features. Two or more test compounds may be added to the aliquots of cells.

Appropriate cells for the present invention include wildtype cells, cells overexpressing a dUTPase, cells overexpressing a uracil-DNA glycosylase, and cells expressing an inhibitor of the uracil-DNA glycosylase function, Ugi (uracil DNA glycosylase inhibitor protein from bacteriohage PBS2). Preferably, these cells are yeast, Homo sapien, D. melanogaster or C. elegans. In yeast cells, the conversion of dUMP to TMP may be inhibited by an antifolate, including aminopterin or sulfanilimide. In another preferred embodiment, the cells overexpress a dUTPase originating from human, animal, plants, fungi, algae, protozoa, bacteria, or a virus. In yet another preferred embodiment, the cells may overexpress a uracil-DNA glycosylase originating from humans, animals, plants, fungi, algae, protozoa, bacteria or virus. Also included in the present invention are cells which in which an inhibitor of uracil-DNA glycosylase is produced, where the cells possess an inhibited uracil-DNA glycosylase function. This inhibitor of uracil-DNA glycosylase may be obtained from a virus (bacteriophage PBS2).

Features measured of the exposed cells for the above methods comprise cell growth or viability, cell cycle checkpoint arrest, presence of replication intermediates in the cells, amount of dUTP present in the cells, and presence or amount of uracil in DNA of the cells. The measured features are interpreted by examining a profile of the cell types. In wild type cells, measured features include cytotoxicity, cell cycle arrest at G1/S or early S-phase, presence of replication intermediates, elevated dUTP pools, or little or no detectable uracil in the DNA. In cells that overexpress dUTPase, measured features include continued growth resistance to cytotoxicity, cell cycle arrest not present or, if present, occurring at early to mid S phase, presence or absence of replication intermediates, low dUTP pools, or little to no detectable uracil in the DNA. In cells which overexpress uracil-DNA glycosylase, measured features include cytotoxicity or enhanced cytotoxicity, cell cycle arrest at G1/S or early S-phase, presence of replication intermediates, elevated dUTP pools, or little to no detectable uracil in the DNA. In Ugi expressing cells, where uracil-DNA glycosylase is inhibited, measured features include enhanced resistance to cytotoxicity, cell cycle arrest at G2/M phase, reduced presence of replication intermediates, elevated dUTP pools, or stable uracil incorporation into DNA.

Further included in the present invention is a kit comprising aliquots of suitable cells and instructions for using the cells in an assay to determine if a test compound induces uracil misincorporation into DNA.

Still further included is a method for determining effectiveness in a patient of chemotherapy targeting conversion of dUMP to TMP. This method includes obtaining a sample of cells from the patient, in which the cells are the target of the chemotherapy. The cells are measured for one or more features, including cell growth or viability, cell cycle checkpoint arrest, presence of replication intermediates in the cells, amount of dUTP present in the cells, or presence or amount of uracil in DNA of the cells.

Various features and advantages of the present invention will be understood by reference to the drawings, detailed description and examples that follow.

### BRIEF DESCRIPTION OF THE DRAWINGS

FIG. 1 is a partial schematic diagram of de novo thymidylate metabolism. FIG. 1A depicts the role of dUTPase in de novo thymidylate biosynthesis. FIG. 1B depicts the mechanism of chemotherapeutic cytotoxicity resulting from inhibitors of thymidylate metabolism.

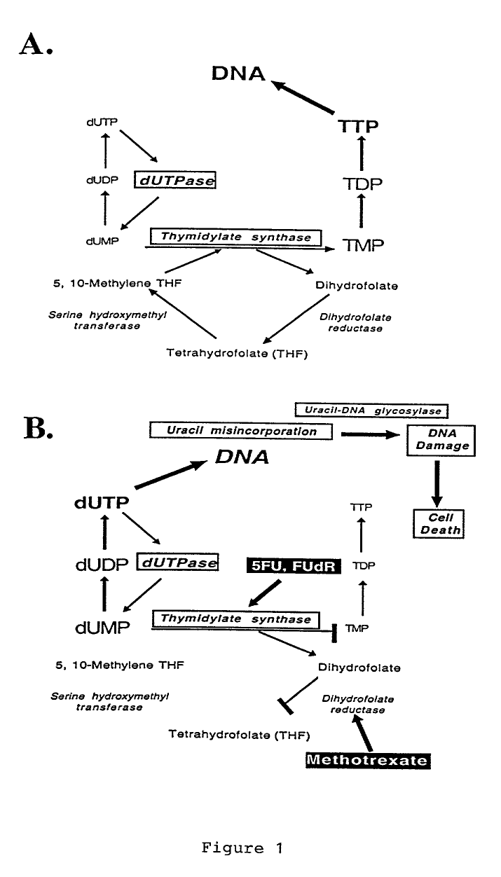

FIG. 2 shows changes in cellular morphology and affects on growth and viability due to antifolate treatment.

FIG. 3 shows overexpression profiles of dUTPase, UDG, and Ugi.

FIG. 4 depicts the effects of antifolates on viability.

FIG. 5 shows TTP and dUTP pool analysis of antifolate treated cells.

FIG. 6 shows detection of replication intermediates and uracil misincorporation into DNA by PFGE.

FIG. 7 is a Fluorescence Activated Cell Sorting (FACS) analysis of asynchronous cultures.

FIG. 8 shows the variability in the completion of DNA replication after transient antifolate treatment is induced by components of uracil-DNA metabolism.

FIG. 9 is a schematic diagram of a model for the effect of aberrant dUTP metabolism on DNA replication and cell cycle control.

### DETAILED DESCRIPTION OF THE INVENTION

As described in greater detail below, this invention provides assays and other methods that arise from the inventors discovery that aberrant uracil-DNA metabolism is the primary mediator of cytotoxicity induced by chemotherapeutic agents that inhibit de novo thymidylate metabolism in Sacchraromyces cerevisiae. This was elucidated by several experiments utilizing a yeast model system, and as set forth in Examples 1-7. Those experiments demonstrate three distinct checkpoints by which yeast can respond to thymineless conditions. The antifolates aminopterin and sulfanilamide induce cytotoxicity by inhibiting the synthesis of the DNA precursor thymidylate. Thymineless death in yeast has been associated with increased recombination, S-phase arrest and cytotoxicity, presumed to be the result of uracil misincorporation and catastrophic repair.

Yeast synchronized in GI and released into media containing antifolates arrest in early S-phase. These cells demonstrate the anticipated TTP pool depletion, dUTP pool expansion and the accumulation of replication intermediates, all correlating with severe cytotoxicity.

A strain overexpressing dUTPase prevents antifolate induced dUTP pool expansion and uracil misincorporation into DNA. This strain demonstrates depleted TTP pools, stalled replication and arrest in early to mid S-phase. When released from drug, this strain exhibits near complete recovery of viability.

Inhibition of the repair enzyme uracil-DNA glycosylase by the inhibitor protein Ugi, results in the stable misincorporation of uracil into DNA upon antifolate treatment. This strain bypasses early and mid S-phase checkpoints and arrests in G2/M. These cells exhibit near complete uracil replacement of thymidine and enhanced short-term viability when released from drugs.

These data demonstrate that thymineless death in yeast is primarily dependent on uracil misincorporation and detrimental repair. Cells can bypass this toxicity through the action of dUTPase. Stable misincorporation of uracil into DNA enables S-phase checkpoint bypass, initiating a G2 checkpoint that is activated in cells that possess Ugi-inhibted uracil DNA glycosylase.

As used herein, the term, “wildtype” refers to a given strain of organism, or to a genotypic or phenotypic trait in a given organism, which is characteristic of the majority of naturally occurring members of its kind, i.e., a non-mutant strain or trait. For purposes of the present invention, the term, “over-expressing” refers to expression of a protein in an amount that is more than that found in the naturally occurring cell of the same type.

As used herein, “antifolate” or “folic acid antagonist” refers to an agent which acts as a structural analogue of folic acid and which binds to and inhibits the enzyme dihydrofolate reductase, and thereby inhibiting the tetrahydrofolate (THF)-dependent reactions (e.g., synthesis of deoxythymidine and hence of DNA).

The term, “aliquot” refers to a sample of predetermined size or fraction, taken from the whole. The actual size or fraction used in a particular aliquot depends on the characteristics of the experiment, but would be understood to one of ordinary skill in the art and determinable without undue experimentation.

As used herein, the term, “chemotherapy” generally refers to the use of drugs for the treatment of diseases including, but not limited to, cancer.

The information set forth herein defines a variety of methods to rapidly and specifically screen for chemotherapeutic agents that induce cytotoxicity through uracil misincorporation into DNA. The methods are not limited to humans, but may be adapted to screen for antiviral and antimicrobial agents as well.

Thus, a broad aspect of the present invention is a method for screening chemotherapeutic agents that induce uracil misincorporation into DNA. In a preferred embodiment, the screening method is based on a series of yeast strains that either over-express enzymes that are involved in uracil DNA mediated cell death or have inactivated, inhibited enzymes or have the gene for these enzymes disrupted or ablated. Specifically, enzymes such as dUTPase, which regulates cellular dUTP pools, and uracil-DNA glycosylase (UDG, which initiates the uracil-specific base-excision repair pathway), are manipulated. The method is based on differential sensitivities of these strains to chemotherapeutic agents. This method also relies on the differential induction of key biochemical hallmarks of uracil-DNA-mediated cytotoxicity; for example, G1, S and G2-specific cell cycle arrest, the induction of DNA replication intermediates, the accumulation of dUTP pools, and the presence of uracil residues in DNA.

These methods are not restricted to yeast or human enzymes but can be used to screen any species enzymes such as those of other mammals, or fungi, plants, protozoa, bacteria and viruses. Although this screen was originally developed in budding yeast, its is not limited to yeast and could be performed within other genetically amenable species, such as D. melanogaster or C. elegans.

A preferred embodiment of the invention utilizes the antifolate agents, aminopterin and sulfanilamide, and employs the following strains of yeast:

1) a wild type yeast strain

2) a strain that overexpresses dUTPase

3) a strain that overexpresses uracil-DNA glycosylase, and

4) a strain that overexpresses the UDG inhibitor protein, Ugi and therefore has a compromised uracil-DNA glycosylase (UDG) function.

The strains are treated with the antifolate agents and assayed for one or more of: a) cell growth/viability, b) cell cycle checkpoint arrest, c) replication intermediates, d) dUTP pool levels and e) the presence of uracil in DNA.

The following results are anticipated if the chemotherapeutic agent induces uracil misincorporation into DNA:

1) wild type strain:

a) cytotoxicity

b) cell cycle arrest at G1/S or early S-phase

c) presence of replication intermediates

d) elevated dUTP pools

e) little to no detectable uracil in DNA

2) dUTPase overexpression strain:

a) enhanced resistance to cytotoxicity

b) cell cycle arrest at early to mid S-phase

c) presence of replication intermediates

d) low dUTP pools

e) little to no detectable uracil in DNA

3) uracil-DNA glycosylase overexpression strain

a) cytotoxicity or enhanced cytotxicity

b) cell cycle arrest at G1/S or early S-phase

c) presence of replication intermediates

d) elevated dUTP pools

e) little to no detectable uracil in DNA

4) Ugi expression strain

a) enhanced resistance to cytotoxicity

b) cell cycle arrest at G2/M phase

c) reduced replication intermediates

d) elevated dUTP pools

e) stable uracil misincorporation

This screen can be performed as a high throughput method by first taking into account growth inhibition and cell cycle status. For example, growth inhibition can be monitored in a 96 well format by measuring culture densities. Once promising compounds are identified, cell cycle analysis by fluorescence activated cell sorting (FACS) can be performed to identify cell cycle status. Agents that induce uracil into DNA are readily identified if they are able to induce the pattern of cell cycle checkpoints as described above. The enzymes dUTPase and UDG confer the extreme specificity of this assay. These enzymes recognize the uracil base exclusively, thereby eliminating alternative mechanism of drug action (e.g. false positives). In addition, the characteristic checkpoint induction of each strain is unique and therefore ensures specificity (e.g., G2 checkpoint in the Ugi strain). Further biochemical characterization of replication intermediates, nucleotide pools and uracil detection in DNA can be subsequently performed to confirm the mechanism of drug action once promising compounds have been identified.

Rapid screening for inhibitors of dUTPase can also be performed using this method. The results of the strain screen may be anticipated to be as follows:

1) Wild type strain:

a) Cytotoxicity

b) Cell cycle arrest in G1/S or early S-phase

c) Presence of replication intermediates

d) Elevated dUTP pools

e) Little to no detectable uracil in DNA

2) dUTPase overexpression strain:

a) Continued growth and resistance to cytotoxicity

b) Either no arrest, or arrest in mid S-phase

c) Possible presence of replication intermediates

d) Low dUTP pools

e) Little to no detectable uracil in DNA

3) Uracil-DNA glycosylase overexpression strain

a) Cytotoxicity or enhanced cytotxicity

b) Cell cycle arrest at G1/S or early S-phase

c) Presence of replication intermediates

d) Elevated dUTP pools

e) Little to no detectable uracil in DNA

4) Ugi expression strain

a) Enhanced resistance to cytotoxicity

b) Cell cycle arrest at G2/M-phase

c) Reduced replication intermediates

d) Elevated DTP pools

e) Stable uracil UTP in DNA

This assay could be used to specifically screen, identify and develop inhibitors against duties from virtually any species, simply by substituting that UTPase; duties encoding DNA in the yeast system. Many outpaces from a wide variety of organisms have been cloned (for a non exhaustive list, see Macintosh & Harness, Act Biochemical Policing 44: 159-172, 1997). We have successfully replaced the yeast duties gene with the human duties enzyme. This suggests that any UTPase; duties enzyme could complement the otherwise lethal DUTY knockout. The basis of this concept could be used for any enzyme that affects UT1 metabolism.

It will be appreciated by one skilled in the art that differential screens of agents against a battery of strains that possess duties enzymes from differing species can be performed to determine if the agent of interest is selective in its activity from species to species.

A number of additional utilities are contemplated for the screening methods of the present invention. For instance, the methods also may be used to determine synergy between agents and to define the mechanism of synergy if related (directly or indirectly) to uracil UTP into DNA. In addition, this and similar screening methods may be used to identify interacting modulators (enzymatic or biochemical) of the uracil-misincorporation pathway. Furthermore, the methods may be used to identify inhibitors or activators (chemical or biological) that repress or induce uracil UTP into DNA.

Therapeutic agents identified using the methods of the invention will be useful for any application in which antiproliferation agents are currently used. Examples include, but are not limited to, anti-cancer agents and treatment for rheumatoid arthritis. Furthermore, depending on the species to which the assays are adapted, therapeutics identified will be useful as anti-viral, anti-parasitic, and anti bacterial agents.

Significantly, in connection with the present invention the inventors have also discovered that stable uracil UTP into DNA leads to a G2 checkpoint arrest. Identification of this DNA lesion (uracil) that induces a G2 checkpoint may reveal additional strategies and targets for chemotherapeutic development. For example, combined inhibition of duties and UDG would lead to the stable incorporation of uracil into DNA. This may lead to G2 arrest and cell death by a mechanism that could be therapeutically beneficial. From the foregoing discussion, it is clear that the discoveries made in accordance with the present invention will provide a significant benefit and advance to the art of identifying and developing new and useful therapeutic agents for modulating cell proliferation. One of skill in the art will also appreciate that these discoveries can be put to use in a variety of diagnostic or prognostic applications. That is, the functional status of one or more of the key enzymes in DTP metabolism may be specifically determined by subjecting cells of a patient to one or more of the assay methods described above. In this manner, a patient's response to chemotherapy may be assessed, or underlying causes of a lack of response to the therapy may be identified. One particular example of the diagnostic/prognostic utility of the invention is in the treatment of leukemia. Leukemias are often treated with dihydrofolate reductase inhibitors, such as methotrexate. To monitor a patient's response to such treatment, white blood cells are isolated from the patient and the cells are subjected to one or more of the assay methods of the invention (i.e., FACS assessment of cell cycle arrest at specific checkpoints) to determine the existence and amount of uracil UTP, as a measure of the effectiveness of the therapy.

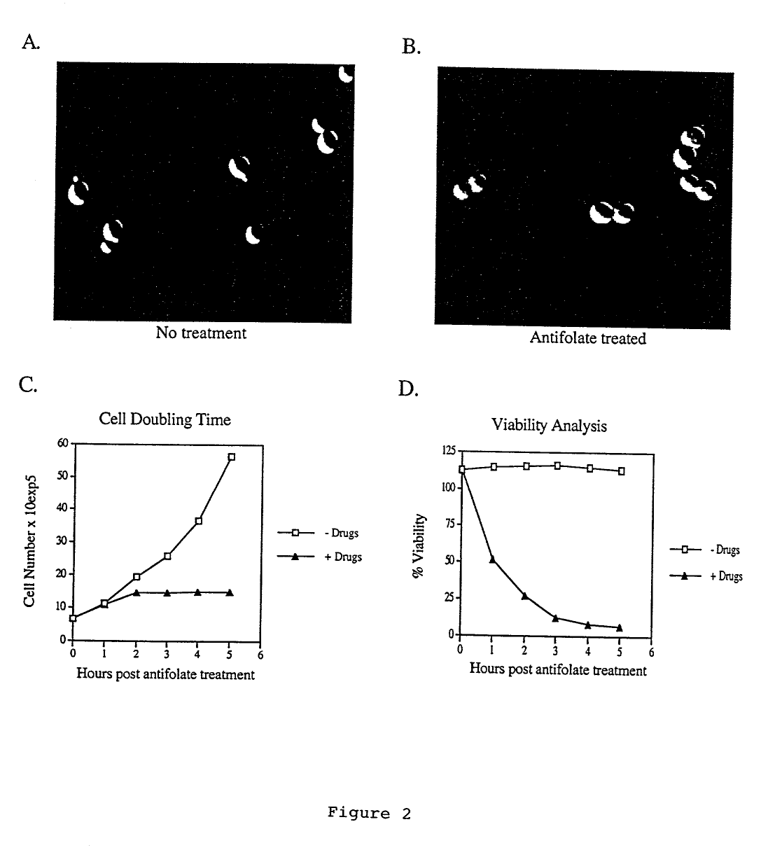

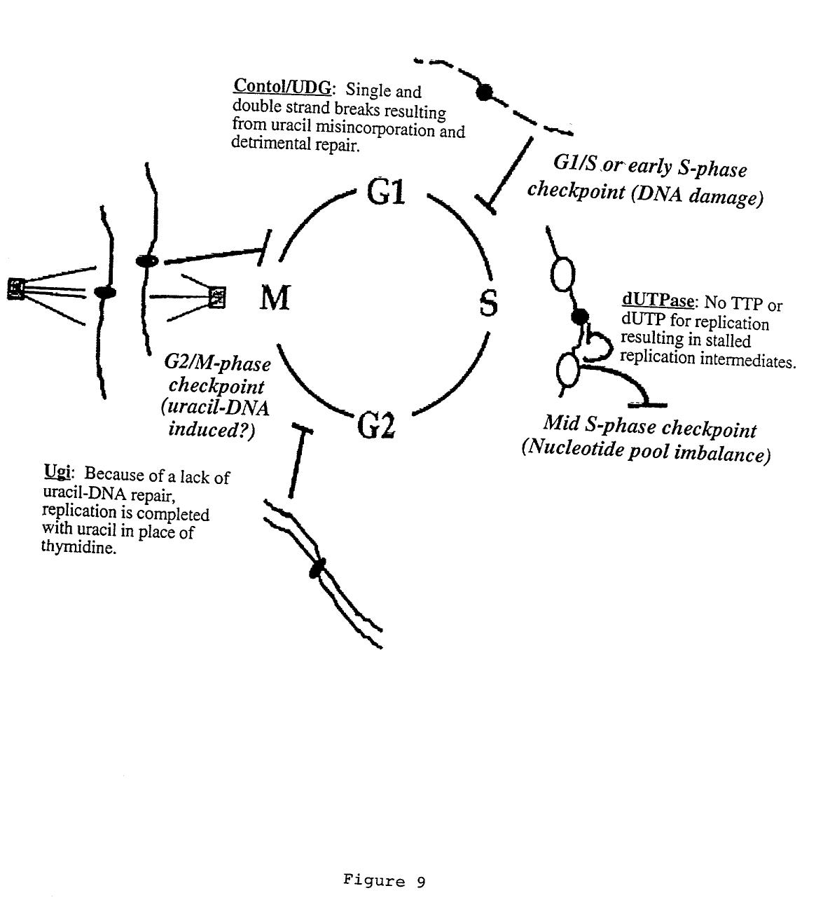

The present invention, as shown in FIGS. 2-8 and in the examples below, demonstrates that uracil UTP into DNA and UDG-mediated repair is the primary mechanism of antifolate action in yeast. The present invention also demonstrates that TTP pool depletion alone is not sufficient to induce cytotoxicity. As shown with data obtained with the control and UDG expression strains, antifolate treatment induces severe cytotoxicity and is correlated with depletion of TTP, the accumulation of DTP, and a DNA damage-dependent G1/S checkpoint arrest due to single and double strand breaks resulting from uracil UTP and detrimental repair. The duties overexpression strain effectively rescues viability and is correlated with TTP and DTP pool depletion and cell cycle arrest in mid S-phase due to nucleotide pool imbalance. The Ugi strain exhibited an enhanced viability that correlates with TTP depletion and DTP accumulation, stable uracil UTP into DNA, and initiation of a G2/M phase checkpoint that may be associated with residual UT1 glycosylase function that initiates the more sensitive G2/M DNA damage checkpoint. FIG. 9 provides a schematic representation of this model.

### EXAMPLES

The following examples are provided to describe the invention in greater detail. They are intended to illustrate, not to limit, the invention.

### Example 1

### Effects of Antifolate Treatment on Yeast Morphology, Growth and Viability

Thymidylate synthase from Saccharomyces cerevisiae requires N5, N10 methylenetetrahydrofolate (THF) as a methyl group donor for the conversion of dUMP to TMP. Concurrent treatment with the antifolates aminopterin and sulfanilamide inhibits thymidylate metabolism by depleting available THF pools. To establish a yeast model of thymineless death, initial experiments were performed to confirm the phenotypes of thymidine starvation induced by antifolates as previously described by Kunz, et al., (1980) Proc. Natl. Acad. Sci. USA 77(10): 6057-6061. Yeast cells in the logarithmic phase of growth were suspended in minimal media containing 100 μg/ml aminopterin plus 5 mg/ml sulfanilamide and incubated at 34° C. with shaking. Time course experiments were performed during drug treatment to assess cellular morphology, growth inhibition and viability.

The changes in cellular morphology due to antifolate treatment are presented in FIG. 2. Culture samples were harvested at 0 and 4 hours post-drug addition and cells were examined by differential interference microscopy. The untreated cells at 0 hour time point (panel A) exhibit normal cellular morphology including unbudded cells and cells with both small and large daughter buds. In contrast, cells treated with drug for 4 hours displayed a “dumbbell morphology”, as illustrated in panel B. Induction of this morphology by antifolates is characteristic of thymidine starvation and DNA synthesis arrest as previously reported (Little and Harness, (1979) Mol. Gen. Genet. 168: 141-151).

Treatment with antifolates also resulted in growth arrest and loss of viability. Results of these experiments are presented in FIG. 2C&D. During a time course of drug treatment, cells were withdrawn, and a determined number of cells were plated on YPD agar to determine viability. Cell growth arrest was observed after 2 hours (panel C) and cell killing was seen within one hour of drug treatment (panel D). The growth arrest and loss of viability observed resembles thymineless death kinetics demonstrated in dTMP auxotrophs (Barclay and Little (1978) Molec. Gen. Genet. 160: 33-40).

Thus, the present invention demonstrates an induction of thymineless death by aminopterin and sulfanilamide as determined by physical morphology, cell growth arrest and loss of viability. In addition, the present invention shows the utility of using yeast as a preferred model system to study thymineless death.

### Example 2

### Cloning and Expression of Duties, Uracil-DNA Glycosylase and the Uracil-DNA Glycosylase Inhibitor Protein in Yeast

To determine how enzymes central to the mechanism of uracil DNA-mediated cytotoxicity affect cell killing under thymineless conditions, we have generated a series of yeast constructs that over-express duties, UT1 glycosylase (UDG), and the UT1 glycosylase inhibitor protein (UGI) which is encoded by the Bacillus subtilus bacteriophage PBS2.

The open reading frame of the yeast duties gene (DUTY) was PCR amplified from total genomic DNA utilizing the following synthetic primers: upper: 5′-CGGATCCATGACTGCT ACTAGCGACAAAG-3′ (SEQ ID NO:1), lower: 5′-CCCAAGCTTTTAGTTACCAGTGCTACCA-3′ (SEQ ID NO:2). Restriction endonuclease sites were incorporated into the sequences of both 5′ (BamHI) and 3′ primers (HindIII) to facilitate cloning. The resulting 460 bp product was gel purified, digested with BamHI and HindIII, ligated into pGEM-3Z and the DUTY sequence verified by DNA sequencing. The DUTY open reading frame was subcloned into the yeast shuttle/expression vector YEp352/GAL1. An 830 bp fragment containing the GAL1 promoter was previously inserted into the EcoR1/HindIII sites of YEp352, thus generating YEp352/GAL1. DUTY was subcloned into the BamHI/HindIII sites that positions the gene just downstream of the GAL1 promoter.

The open reading frame yeast UT1 glycosylase gene (UNG1/UDG) was PCR amplified from total genomic DNA utilizing the following synthetic primers: upper: 5′-CGGATCCATGTGGTGCATGAGAAGATTGC-3′ (SEQ ID NO:3), lower: 5′ CCCAA GCTTTCAAGGGTCCTTTGATTCTGACTC-3′ (SEQ ID NO:4). Restriction endonuclease sites were added to both 5′ (BamHI) and 3′ primers (HindIII) to facilitate cloning. The resulting 1.1 kb product was gel purified, digested with BamHI and HindIII, ligated into pGEM-3Z and the UDG sequence verified by DNA sequencing. The UDG open reading frame was subcloned into the yeast shuttle/expression vector YEp352/GAL1 (BamHI/HindIII sites).

The open reading frame of the bacteriophage PBS2-encoded UT1 glycosylase inhibitor protein (Ugi) was PCR amplified from the previously cloned gene (Caradonna et al., 1996) utilizing the following synthetic primers: upper: 5′ CGGGATCCATGACAAATTTATCTGACATCATTG-3′ (SEQ ID NO:5), lower: 5′ CCCAAG CTTATAACATTTTAATTTTATTTTCTCCATTAC-3′ (SEQ ID NO:6). Restriction endonuclease sites were added to both 5′ (BamHI) and 3′ primers (HindIII) to facilitate cloning. The resulting 270 bp product was gel purified, digested with BamHI and HindIII, ligated into pGEM-3Z and the Ugi sequence verified by DNA sequencing. The UDG open reading frame was then subcloned into the yeast shuttle/expression vector YEp352/GAL1 (BamHI/HindIII sites).

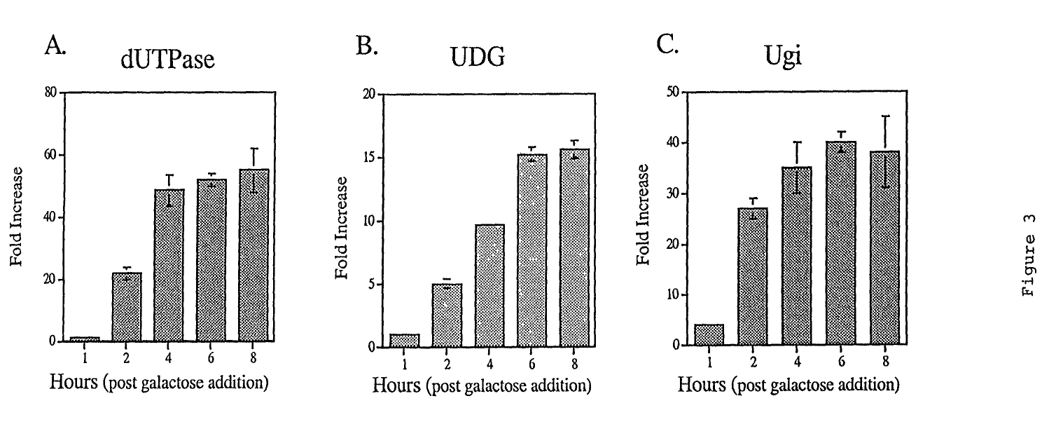

All three expression constructs, including a negative control vector containing no insert, were transformed into W303 cells utilizing a lithium acetate-based protocol as described by Geitz and Schiestl (1995). Cells containing each construct were grown in minimal-selective media (URA drop out to select for YEp352) containing 2% raffinose, which is a non-repressing carbon source for the GAL1 promoter. The proteins were induced to express by transferring the cells into minimal-selective media containing 2% galactose. Expression of each protein was monitored by enzyme activity in total cellular extracts. The in vitro assays for duties and UDG are established in our laboratory and have been previously described Caradonna et al. (1996) Experimental Cell Res. 222: 345-359. The activity of Ugi was determined by assessing the amount of UDG activity that was inhibited in mixing experiments. Time points were taken at 0, 2, 4, 6 and 8 hours post-galactose stimulation, extracts were prepared, protein concentrations equilibrated, and enzyme activities determined. The results of these experiments are shown in FIG. 3. The data presented represents the average of three independent experiments and the enzyme activities are expressed as fold-increase over the uninduced state.

The increase in duties, UDG, and Ugi activities are illustrated in panels A, B and C respectively. The doubling times of these strains in the induced state are identical to the control strain that contains plasmid with no insert. Expression of duties is increased 50-fold over normal cellular activity after 8 hours of induction (panel A). Later time points (12 and 24 hours) suggest that this 50-fold increase duties activity represents a steady-state level when cells are under a continued state of induction by galactose. UDG activity is increased over 15-fold over normal cellular activity after 8 hours of induction (panel B). Analysis of UDG activity over a longer time course suggests that the 15-fold increase represents a steady state level over 24 hours.

Ugi activity is increased 40 fold over non-induced levels. It is important to note that Ugi expression effectively inhibits all cellular UDG activity even in the non-induced state (2% raffinose). It is assumed that the GAL1 promoter is “leaky” and even this low level of Ugi expression is sufficient to inhibit normal cellular levels of UDG in yeast. The increase in Ugi activity presented in panel C represents the fold induction over the non-induced state. These experiments were performed by mixing diluted cellular extracts from the Ugi expressing strain with cellular extracts from the control strain (YEp352/GAL1 with no insert).

These studies demonstrate the successful expression of duties, UDG and Ugi from the galactose inducible promoter GAL1 in yeast. Successful construction of these strains establishes a model system to assess the role of these central enzymes in the underlying molecular mechanism of thymineless death in yeast.

### Example 3

### Effect of duties, UDG and UGI Expression on Sensitivity to Aminopterin and Sulfanilamide

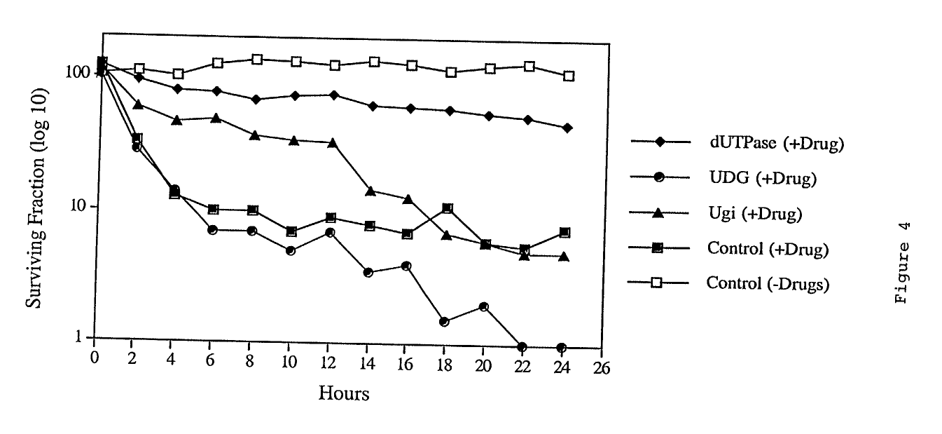

To address the influence of duties, UDG and Ugi expression on sensitivity to antifolate treatment, we performed viability assays over a 24 hour time course. Cultures were grown for 12 hours in minimal-selective media containing 2% galactose to induce protein expression from the YEp352/GAL1 constructs. Exponentially growing cultures were then seeded at 5×106 cells/ml in media containing 100 μg/ml aminopterin plus 5 mg/ml sulfanilamide or media without drugs and incubated at 34° C. with shaking. Cells were harvested at 2 hour time-points, counted and a determined number of cells plated on YPD agar plates. Viability was assessed after three days of colony growth. Results from these experiments are presented in FIG. 4. The values presented in this figure represent averaged values from three independent experiments.

The control strain (YEp352/GAL1/no insert) demonstrated a characteristic loss of viability upon treatment with antifolates. The untreated control strain showed no loss of viability over the 24 hour time course, as did all of the untreated expression strains tested. The duties overepressing strain (YEp352/GAL1/DUT1) demonstrated remarkable resistance to treatment with antifolates throughout the 24 hour time course. The dUTPase-induced resistance correlates to more than 70% greater viability at 6 hrs post-drug treatment when compared to the drug-treated control. This suggests a critical role for duties in modulating sensitivity to antifolates in yeast.

The UDG overexpression strain (YEp352/GAL1/UDG) demonstrated the same loss of viability kinetics as the control strain during the first 12 hours. During hours 14-24 post-drug addition however, there is a greater sensitivity of this strain to antifolate treatment corresponding to a loss of viability. Therefore, an increase in UDG activity may result in greater DNA strand breaks and increased cell killing. However, the increase in UDG activity appears to have no effect at early time points, suggesting that normal cellular levels of UDG are more than sufficient to induce DNA damage that results in cell kill.

The Ugi overexpression strain (YEp352/GAL1/Ugi) induced greater viability (>40%) when compared to the control during the first 12 hours of the time course. However, a rapid loss of viability occurred between hours 12 and 16. After 16 hours of drug treatment, no difference was observed between the viability of the UGI expression strain and the control strain. These data suggest that Ugi mediated inhibition of UDG activity may prevent UT1 repair-mediated degradation resulting in the stable incorporation of uracil into DNA and greater initial viability—a hypothesis in accord with studies performed with dut-/ung- double mutants of E. coli. (El-Hajj et al., 1992). The rapid decline of viability in this strain after 16 hours may suggest that a critical threshold of uracil UTP into DNA may have been reached that leads to the general failure of macromolecular synthesis and cell death.

Thus, the present invention demonstrates that: 1) duties overexpression induces greater resistance to antifolates throughout a 24 hour drug treatment, 2) Ugi overexpression induces greater resistance during the first 16 hours of drug treatment, after which sensitivity to antifolate treatment is identical to the control strain, 3) UDG expression has no effect on viability when compared to controls for the first 12 hours, after which greater sensitivity to antifolate treatment is observed. This is consistent with the model of uracil DNA-mediated cytotoxicity and provide strong evidence for a central role of aberrant UT1 metabolism as a critical mediator of thymineless death in yeast.

### Example 4

### Determination of TTP and DTP Pools in Antifolate Treated Yeast Cells

Although there is a substantial body of genetic evidence pointing to uracil UTP into DNA as a mediator of thymineless death (reviewed by Kunz, et al. (1994) Mutation Res. 3118: 1-64), there have been no biochemical studies to confirm the underlying mechanism of cell killing in yeast. According to one model of cell killing, the inhibition of TMP synthesis induces the depletion of TTP pools and the rapid accumulation of DTP pools. The present example tests this model by utilizing an enzymatic assay to quantitate TTP and

DTP pools from yeast cell extracts.

The nucleotide pool assay is a modification of a DNA polymerase-based assay first described by Shermann and Fyfe (1989) Anal. Biochem. 177: 222-226. Synthetic oligonucleotides were generated that act as template (5′-TTATTATTATTATT ATTAGGCGGTGGAGGCGG-3′ (SEQ ID NO:7)) and primer (5′ CCGCCTCCACCGCC-3′ (SEQ ID NO:8)) in the dNTP analysis. This assay measures TTP levels by quantitating the TTP-dependent incorporation of [H3]dATP into the template primer duplex. DNA polymerases do not discriminate between TTP and DTP during the replication reaction. In order to measure TTP and DTP independently, nucleotide mixtures were pre-digested with duties that eliminates DTP from the polymerization reaction. The expression and purification of recombinant human duties for use in this assay is described in Ladner et al. (1996) J. Biol. Chem. 271: 7745 7751; 7752-7757. Untreated nucleotide extracts were assayed first, providing a value for total TTP+DTP. Identical samples were then pre-incubated with duties and nucleotide pools determined again providing a value for TTP alone. The value for DTP was determined by subtracting measured TTP levels from the value determined for TTP and DTP combined (Horowitz, et al. (1997) Biochem. Pharm. 54: 635-638). For use on yeast cells, trichloroacetic acid was substituted for perchloric acid for making acid-soluble cellular extracts. Perchloric acid was ineffective in providing reproducible results. This assay were linear between 0.5 and 12 picomoles.

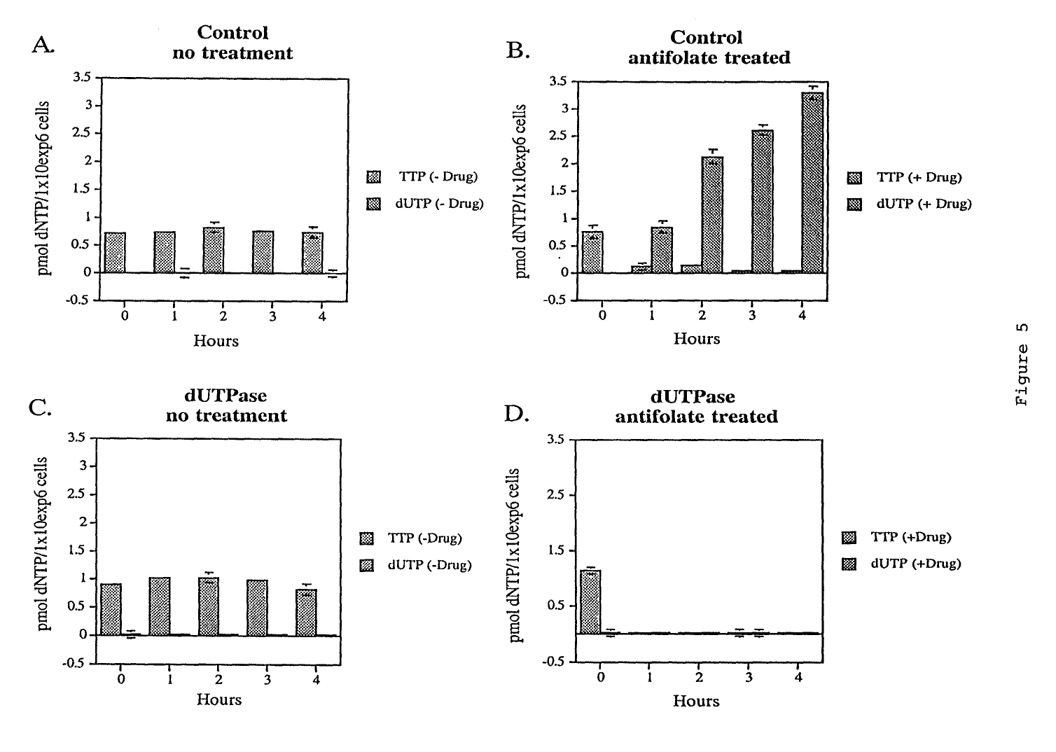

Nucleotide pools were determined in yeast strains that were either untreated or treated with aminopterin and sulfanilamide over a 4 hour time course. Results from experiments performed on the control strain and the duties overexpression strain are illustrated in FIG. 5. After each time point, cells were harvested, washed and DTP and TTP pools were determined by the DNA polymerase-based assay described. Values represent averages from three independent experiments. Untreated cells contained measurable TTP levels (5.2±1.2 pmol/1×106 cells) while DTP was undetectable by this method (panel A&C). In contrast, antifolate treated cells demonstrate a rapid decline in TTP levels within 1 hour of drug treatment, falling to less than 0.1 pmol/1×106 cells within 3 hours (panel B&D). In dramatic contrast, DTP levels were elevated from undetectable levels to 3.3±0.1 pmol/1×106 cells within four hours in the control strain, as shown in panel B. These data indicate that the dUTP:TTP ratio dramatically increases to greater than 33:1 after 4 hours of antifolate treatment in the control strain. The duties overexpression strain demonstrates a complete lack of both TTP and DTP. This suggests that the protective effects of duties overexpression correlate with the prevention of DTP pool accumulation.

Thus, the present invention enables the successful determination of TTP and DTP pool sizes in yeast using a modified DNA polymerase-based nucleotide pool assay. It was shown that TTP pool levels decline after treatment with antifolate drugs, while DTP pools accumulate in the control strain in a time-dependent fashion. The duties overexpression strain demonstrates a lack of DTP pool accumulation. This strongly supports the model of UT1 mediated cytotoxicity, where DTP levels are elevated in response to thymidylate inhibition. In addition, TTP pool depletion alone does not induce thymineless death, which suggests an essential role for aberrant UT1 metabolism in mediating cytotoxicity.

### Example 5

### Detection of Replication Intermediates and Uracil Residues in DNA

The proposed model of UT1 mediated cytotoxicity suggests that when the ratio of dUTP:TTP increases as a result of antifolate treatment, DTP is readily utilized by DNA polymerase in place of TTP and uracil is misincorporated into DNA during replication and repair. To examine this endpoint in yeast, an enzyme-based assay was developed to detect the presence of uracil in DNA. This assay is based on the sequential use of enzymes involved in the uracil-base excision repair pathway. First, yeast cells were prepared for pulsed field gel electrophoresis (PFGE). The resulting agarose plugs containing the yeast DNA were subsequently treated with recombinant UT1 glycosylase (UDG) to generate apyrimidinic sites specifically where dUMP has been misincorporated into DNA. The DNA plug was treated with recombinant AP (apurinic/apyrimidinic) endonuclease (APN1 from yeast). This is a Class II AP endonuclease that cleaves the DNA 5′ to the abasic site resulting in a 3′-OH. DNA was subjected to electrophoresis to visualize and degradation induced the combined action of UDG and APE. Control reactions were performed with no enzyme pre-treatment.

Recombinant UDG expression and purification were described previously by Caradonna et al. 1996, supra. The open reading frame of the major Class II AP endonuclease from yeast (APN1) was PCR amplified from total genomic DNA utilizing the following synthetic primers: upper: 5′-CGG GAT CCA TGC CTT CGA CAC CTA GCT T -3′ (SEQ ID NO:9), lower: 5′-GGG GTA CCT TAT TCT TTC TTA GTC TTC CTC TTC T -3′ (SEQ ID NO:10). Restriction endonuclease sites were added to both 5′ (BamHI) and 3′ primers (KpnI) to facilitate cloning. The resulting 1.1 kb product was gel purified, digested with BamHI and KpnI, ligated into pGEM-3Z and the APN1 sequence verified by DNA sequencing. The APN1 open reading frame was subcloned into the expression vector pProEx Htb using the BamHI and KpnI sites. This vector adds a 6-His tag to the amino termini of the protein to facilitate purification. The protein was successfully over-expressed in DH5a cells, and was readily purified by use of a Ni2+-based purification resin.

Detection of replication intermediates (stalled replication complexes and replication bubbles) was also performed by PFGE. Inhibition of DNA replication by hydroxyurea induces replication intermediates due to a lack of dNTPs for replication. These intermediates are readily detected by their inability to penetrate the agarose gel during PFGE. The abandoned replication forks and replication bubbles have a topology that prevents their entering the gel. Therefore, when chromosomal DNA contains replication intermediates, the DNA remains in the sample well (Hennessy, et al.(1991) Genes and Dev. 5:958-969).

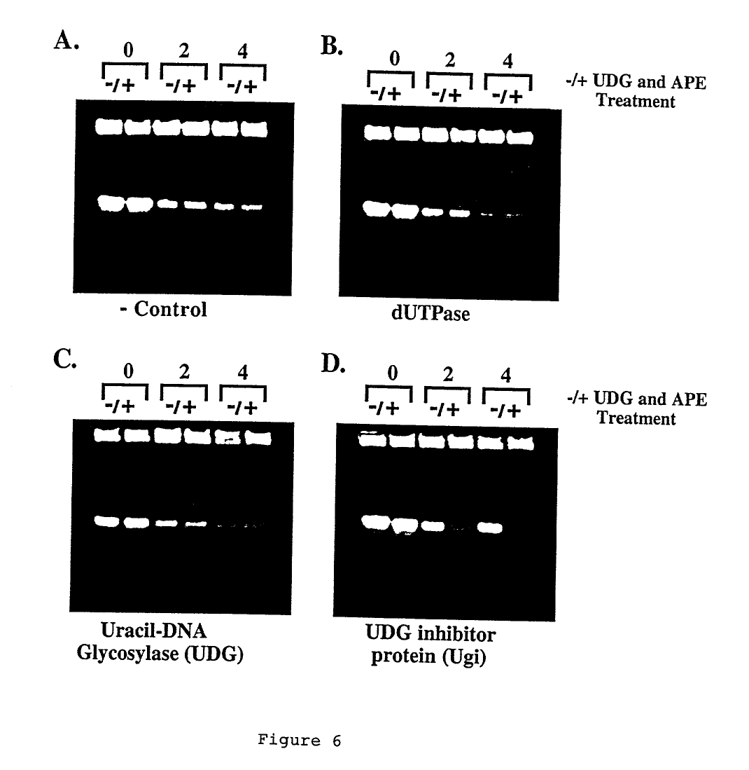

Yeast cells in the exponential phase of growth were treated with aminopterin and sulfanilamide and samples were harvested at 0, 2 and 4 hours after drug addition. After processing for PFGE, agarose plugs containing the yeast DNA were analyzed for replication intermediates and uracil UTP as described. Agarose plugs for each time point was either left untreated (−), or treated with both UDG and APE (+). The plugs were subjected to PFGE. Results of these experiments are show in FIG. 6. The parameters PFGE were altered so that all of the yeast chromosomes ran as a single band. After antifolate treatment, there was a time-dependent decrease in DNA content in the −Control (panel A), duties (panel B) and UDG overexpression lines (panel C). These strains also show no detectable uracil in their DAN as evidenced by no loss of DNA content upon UDG/APE treatment (+lanes). In contrast, the Ugi expression strain (panel D) exhibits almost complete protection from the formation of replication intermediates. Additionally, the DNA derived from the Ugi overexpression strain demonstrates a time dependent increase in uracil content. This was evidenced by degradation of the UDG/APE treated samples (+lanes) at the 2 and 4 hour time points. Thus, Ugi overexpression strain demonstrated the presence of stably misincorporated uracil residues.

Thus, the present invention demonstrates that antifolate treatment induces replication intermediates in the control and duties and UDG overexpression lines. In contrast, the Ugi expression strain lack replication intermediates but did show a stable uracil UTP in its DNA.

### Example 6

### Cell Cycle Checkpoint Analysis by Fluorescence Activated Cell Sorting

(FACS)

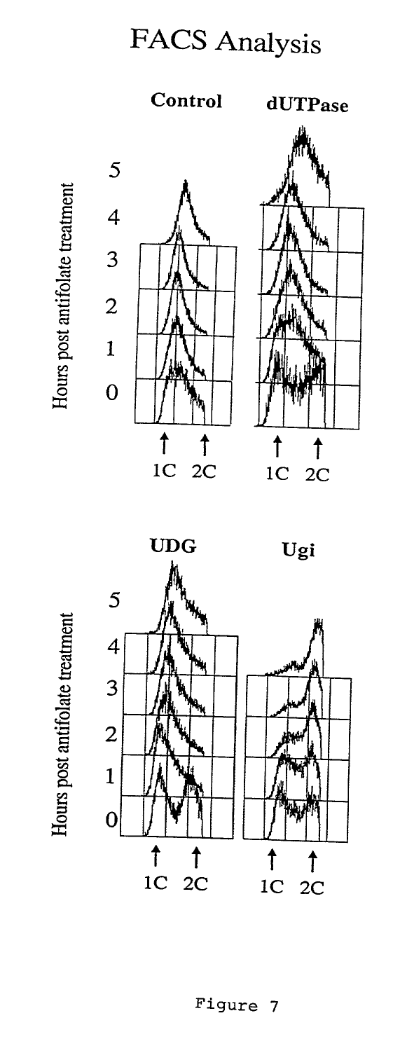

To understand where each of the yeast strains was arresting in the cell cycle post-antifolate treatment, FACS analysis was performed over a five hour time course. Asynchronous cultures were treated with aminopterin and sulfanilamide as previously described and cells were harvested every hour and prepared for FACS analysis. The results of these experiments are presented in FIG. 7. Subsequent to drug treatment, the control strain exhibited a G1/S or early S-phase arrest, as indicated by a near 1C content of DNA. It is assumed that this early S-phase arrest is a DNA damage-induced checkpoint resulting from detrimental uracil UTP and repair. In contrast, the duties overexpression strain bypassed this checkpoint and arrested in mid S-phase (between 1C and 2C content of DNA). The nucleotide pool data indicate that, in this strain, there is no thymidine or uracil for replication. This suggests that mid S-phase arrest is due to nucleotide pool imbalance similar to that observed with hydroxyurea (Desany, et al. (1998) Genes and Dev. 12: 2956-2970). Although the UDG strain is identical to the control, the Ugi overexpression strain strikingly arrests at the G2/M boundary (indicated by a 2C content of DNA). The replication intermediate and uracil UTP data demonstrate that the Ugi overexpression strain completes DNA replication by utilizing uracil in place of thymidine. The G2/M checkpoint arrest is therefore likely due to the presence of uracil in DNA. This constitutes the identification of the first DNA lesion (uracil) that is able to bypass both G1/S and S phase checkpoints while still initiating a DNA-directed G2/M checkpoint (Paulovich, et al. (1997) Cell 88: 315-321).

### Example 7

### DNA Structural Damage Correlates With Viability in Antifolate Treated Strains

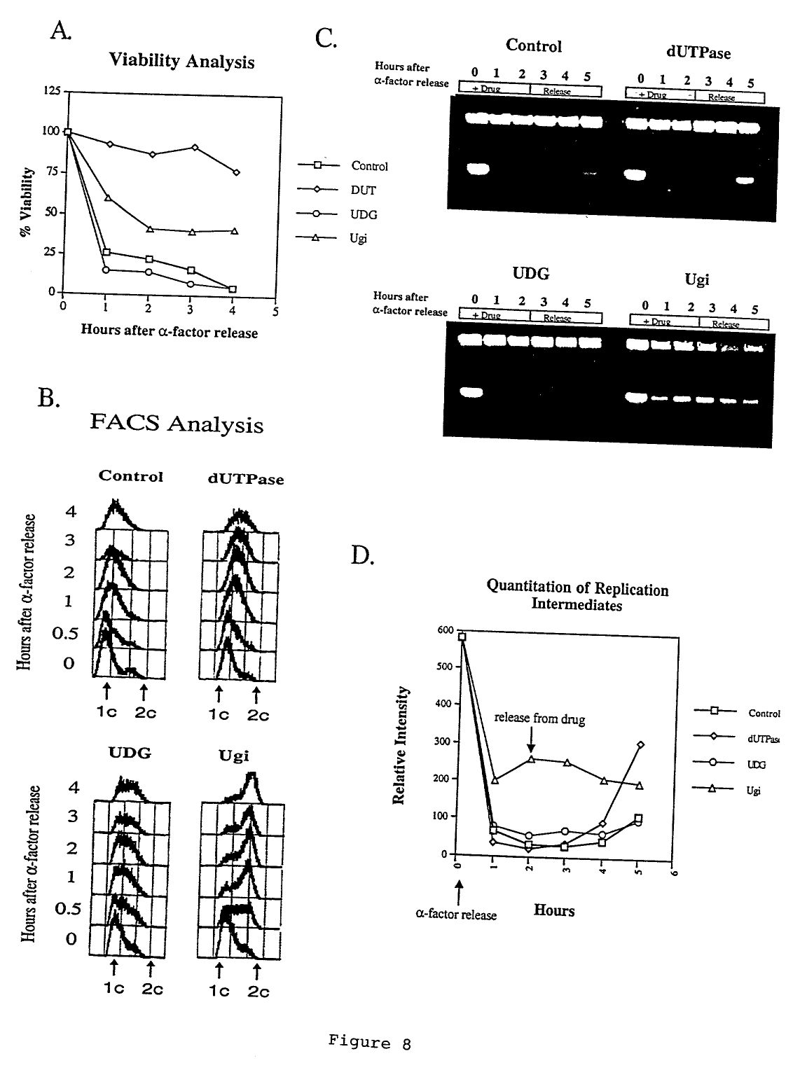

To determine if the cause of cytotoxicity and enhanced protection was associated with the integrity of replicated DNA, we performed a series of experiments that correlate cell cycle status with DNA replication intermediates and viability. Cells were treated with alpha factor to induce a G1 arrest. Cells were released from alpha factor into antifolates and 10 μg/ml nocodazole, the antifolates were washed away after 2 hr, and samples were analyzed for viability and DNA replication intermediates. FIG. 8A demonstrates the characteristic viability profile as previously seen in FIG. 4. The only difference between FIG. 4 and FIG. 8A was that the kinetics of cell kill were much quicker in FIG. 8A due to the cell synchronization. FACS analysis demonstrates the characteristic cell cycle checkpoints observed by each strain as demonstrated in FIG. 8B. Upon treatment with antifolate, the control, UDG and duties strains demonstrated the accumulation of replication intermediates. Upon washout, the duties overexpression strain increased recovery of chromosomal integrity. This increase in chromosomal integrity corresponds to an increased viability of the cells. The control and UDG strains were substantially lacking in the completion of DNA replication. This suggests a profoundly abnormal DNA structure that corresponds to extreme cytotoxicity (Desany, et al., 1998, supra). The Ugi overexpression strain demonstrated significant protection from the induction of replication intermediates. This protection also supports and corresponds to enhanced viability of the cells. The quantitation of these replication intermediates is shown in FIG. 8D.

The present invention is not limited to the embodiments described and exemplified above, but is capable of variation and modification without departure from the scope of the appended claims.

We claim:

1. A method for determining if a test compound induces uracil UTP into DNA, the method comprising:
a) providing aliquots of the following cells:
i) wildtype cells;
ii) cells overexpressing a duties;
iii) cells overexpressing a UT 1 glycosylase; and
iv) cells expressing the UT 1 glycosylase inhibitor protein Ugi or cells possessing a compromised UT1 glycosylase function;
b) exposing the cells to an agent that directly or indirectly inhibits thymidylate metabolism, in the presence or absence of the test compound;
c) measuring one or more features of the exposed cells, the features comprising:
i) cell growth or viability;
ii) cell cycle checkpoint arrest;
iii) presence of replication intermediates in the cells;
iv) amount of DTP present in the cells; and
v) presence or amount of uracil in DNA of the cells; and
d) interpreting the measured features, wherein a profile in the four cell types which is indicative that the test compound induces uracil mis-incorporation into DNA comprises one or more features in each of the cell types comprising:
i) in the wildtype cells, cytotoxicity, cell cycle arrest at G1/S or early S phase, presence of replication intermediates, elevated DTP pools or little or no detectable uracil in the DNA;
ii) in the duties overexpressing cells, enhanced resistance to cytotoxicity, cell cycle arrest at mid S-phase, presence of replication intermediates, low DTP pools, or little to no detectable uracil in DNA
iii) in the UT1 glycosylase overexpressing cells, cytotoxicity or enhanced cytotoxicity, cell cycle arrest at G1/S or early S-phase, presence of replication intermediates, elevated DTP pools, or little to no detectable uracil in DNA; and
iv) in the nonfunctional UT1 glycosylase cells, enhanced resistance to cytotoxicity, cell cycle arrest at G2/M phase, reduced presence of replication intermediates, elevated DTP pools, or stable uracil incorporation into DNA.

2. The method of claim 1, wherein the cells are of an organism selected from the group consisting of yeast, D. melanogaster, and C. elegans.

3. The method of claim 2, wherein the cells are yeast cells and the conversion of dUMP to TMP is inhibited by an antifolate.

4. The method of claim 3, wherein the antifolate is selected from the group consisting of aminopterin and sulfanilimide.

5. The method of claim 1, wherein the cells overexpress a duties from an organism selected from the group consisting of humans, animals, plants, fungi, algae, protozoa, bacteria and viruses.

6. The method of claim 1, wherein the cells overexpress a UT1 glycosylase from an organism selected from the group consisting of humans, animals, plants, fungi, algae, protozoa, bacteria and viruses.

7. The method of claim 1, wherein the cells lacking a UT1 glycosylase function are produced by producing in the cells an inhibitor of UT1 glycosylase.

8. The method of claim 1, wherein the inhibitor of UT1 glycosylase is obtained from a virus.

9. The method of claim 1, adapted for determining if the test compound inhibits duties, the adaptation comprising, in step (d), observing in each of the four cell types one or more features comprising:
i) in the wildtype cells, cytotoxicity, cell cycle arrest at G1/S or early S phase, presence of replication intermediates, elevated DTP pools, or little or no detectable uracil in the DNA;
ii) in the duties overexpressing cells, continued growth resistance to cytotoxicity, cell cycle arrest not present or, if present, occurring at mid S-phase, presence or absence of replication intermediates, low DTP pools, or little to no detectable uracil in DNA;
iii) in the UT1 glycosylase overexpressing cells, cytotoxicity or enhanced cytotoxicity, cell cycle arrest at G1/S or early S-phase, presence of replication intermediates, elevated DTP pools, or little to no detectable uracil in DNA; and
iv) in the UT1 glycosylase inhibitor (Ugi) expressing cells, enhanced resistance to cytotoxicity, cell cycle arrest at G2/M phase, reduced presence of replication intermediates, elevated DTP pools, or stable uracil incorporation into DNA.

10. The method of claim 1, wherein two or more test compounds are added to the aliquots of cells.

11. A kit comprising:
a) aliquots of the following cells:
i) wildtype cells;
ii) cells overexpressing a duties;
iii) cells overexpressing a UT1 glycosylase; and
iv) cells lacking a UT1 glycosylase function; and
b) instructions for using the cells in an assay to determine if a test compound induces uracil UTP into DNA.

12. A method for determining effectiveness in a patient of chemotherapy targeting conversion of dUMP to TMP, the method comprising:
a) obtaining from the patient a sample of cells which are the target of the chemotherapy;
b) measuring one or more features of the cells, the features comprising:
i) cell growth or viability;
ii) cell cycle checkpoint arrest;
iii) presence of replication intermediates in the cells;
iv) amount of DTP present in the cells; and v) presence or amount of uracil in DNA of the cells; and
c) observing if one or more of the measured features is the same as or differs from features comprising: cytotoxicity, cell cycle arrest at G1/S or early S-phase, presence of replication intermediates, elevated DTP pools or little or no detectable uracil in the DNA, wherein a lack of divergence from one or more of the features is indicative that the chemotherapy is effective, and a divergence from one or more of the features indicates a possibility that the chemotherapy is of reduced effectiveness or is ineffective.


SEQUENCE LISTING (10 sequences)

SEQ ID NO: 1 - DNA, 29 bp, Artificial Sequence
  Saccharomyces cerevisiae synthetic oligonucleotide
cggatccatgactgctactagcgacaaag

SEQ ID NO: 2 - DNA, 28 bp, Artificial Sequence
  Saccharomyces cerevisiae synthetic oligonucleotide
cccaagcttttagttaccagtgctacca

SEQ ID NO: 3 - DNA, 29 bp, Artificial Sequence
  Saccharomyces cerevisiae synthetic oligonucleotide
cggatccatgtggtgcatgagaagattgc

SEQ ID NO: 4 - DNA, 33 bp, Artificial Sequence
  Saccharomyces cerevisiae synthetic oligonucleotide
cccaagctttcaagggtcctttgattctgactc

SEQ ID NO: 5 - DNA, 33 bp, Artificial Sequence
  Saccharomyces cerevisiae synthetic oligonucleotide
cgggatccatgacaaatttatctgacatcattg

SEQ ID NO: 6 - DNA, 39 bp, Artificial Sequence
  Saccharomyces cerevisiae synthetic oligonucleotide
cccaagcttataacattttaattttattttctccattac

SEQ ID NO: 7 - DNA, 32 bp, Artificial Sequence
  Saccharomyces cerevisiae synthetic oligonucleotide
ttattattattattattaggcggtggaggcgg

SEQ ID NO: 8 - DNA, 14 bp, Artificial Sequence
  Saccharomyces cerevisiae synthetic oligonucleotide
ccgcctccaccgcc

SEQ ID NO: 9 - DNA, 28 bp, Artificial Sequence
  Saccharomyces cerevisiae synthetic oligonucleotide
cgggatccatgccttcgacacctagctt

SEQ ID NO: 10 - DNA, 34 bp, Artificial Sequence
  Saccharomyces cerevisiae synthetic oligonucleotide
ggggtaccttattctttcttagtcttcctcttct